(19) United States
(12) Patent Application Publication
(10) Pub. No.: US 2016/0199384 A1
(43) Pub. Date: Jul. 14, 2016

(54) Method and Topical Composition for the Treatment of Rosacea and Skin Erythema Using Pyrithione Zinc

(71) Applicant: Paul Sutich, Long Branch, NJ (US)

(72) Inventor: Paul Sutich, Long Branch, NJ (US)

(21) Appl. No.: 14/594,735

(22) Filed: Jan. 12, 2015

(51) Int. Cl. A61K 31/555; A61K 9/00

(52) U.S. Cl. CPC A61K 31/555; A61K 9/0014

(57) ABSTRACT

A method for the treatment of Rosacea and skin erythema using pyrithione zinc as an active ingredient in a topically applied administration to a user's skin.

### CROSS-REFERENCE TO RELATED APPLICATIONS

None.

### STATEMENT REGARDING FEDERALLY SPONSORED RESEARCH OR DEVELOPMENT

None.

### REFERENCE TO A MICROFICHE APPENDIX

None.

### BACKGROUND OF THE INVENTION

1. Field of the Invention

This invention relates to the treatment of Rosacea and skin erythema, in general, and to a method utilizing a topical composition employing pyrithione zinc of different concentrations depending on the severity and extent of user symptoms, in particular.

2. Description of the Related Art

Rosacea is a very common red, acne-like benign skin condition that affects approximately 45 million people worldwide. Its main symptoms include red or pink facial skin, small dilated blood vessels, small red bumps sometimes containing pus, cysts, and pink or irritated eyes. Characteristically involving the central region of the face (mainly the forehead, the cheeks, the chin, and the lower half of the nose), Rosacea is considered a chronic, long-term incurable skin condition with periodic ups and downs. Tending to occur more frequently in women, but more severely in men, Rosacea strikes both at potentially all ages. Unfortunately, emotional factors such stress, fear, anxiety and embarrassment have been determined to aggravate the condition.

While not considered contagious or infectious—and even not necessarily requiring treatment if the individual is not bothered by the condition—many treatment choices are available depending on the severity and extent of symptoms. Antibacterial washes, topical medications, oral antibiotics, Lasers, pulsed-light therapies, photodynamic therapy, and isotretinoin have oftentimes been utilized—with varying degrees of success and commensurate costs. Combination uses of them are not uncommon, with some being utilized alternatingly in the morning and at night, and others, such as with topical medications, more than once or twice a day. With metronidazole topical antibiotic medication (Rosadan®) and Azelaic Acid (Finacea®) costing in prescription amounts by dermatologists of $300.00 and more, it would be advantageous to have a simple pharmaceutical composition to effectively overcome the flushing and redness in the case of Rosacea, which is not yet achievable by the use of available treatments at a less expensive cost.

As will be understood, the same holds true for skin erythema, in general.

### OBJECTS OF THE PRESENT INVENTION

It is an object of the present invention, therefore, to provide an effective composition for the treatment of Rosacea.

It is an object of the present invention, also, to provide such a composition which is both easy to apply, and which can be obtained at a more affordable price.

It is an additional object of the invention to provide such a composition which for mild instances of Rosacea may be obtained over-the-counter, without the need for prescription.

It is a further object of the invention to provide a composition of greater concentration for a more resistant condition of Rosacea, obtainable by prescription.

It is yet a further object of the present invention to provide such a composition for treating other conditions of the skin characterized by redness (i.e. skin erythema), and either as a gel, cream or lotion.

### SUMMARY OF THE INVENTION

As will become clear from the following description, the objects of the present invention are achieved through the employment of zinc pyrithione as an active ingredient in the method of treatment. As will be understood by those skilled in the art, available in white-to-slightly yellow crystals and commercially supplied as a 24-26% aqueous solution, zinc pyrithione is also known by the following chemical names Bis[1-hydroxy-2(1H)-pyridinethionato-O,S] (T-4) zinc (IUPAC), pyrithione zinc, Zinc bis(2-pyridylthio)-N-oxide), Zinc pyridinethione, Zinc 2-pyridinethione-1-oxide, Bis (N-oxopyridine-2-thionato) zinc (II), ZP, ZnPT, ZnPTO, BOTZ; and under the tradenames Zinc Omadine and Vancide ZP—and also having the empirical formula C₁₀H₈N₂O₂S₂Zₙ. In a tested embodiment of the invention, the zinc pyrithione is combined with a cocoa butter moisturizer carrier in a treatment of the Rosacea. In a preferred embodiment, pyrithione zinc is employed as the sole active ingredient in the treatment.

### DETAILED DESCRIPTION OF THE INVENTION

Zinc pyrithione is currently regulated as a preservative in rinse-off products (excluding oral hygiene products) in an concentration up to 0.5% in general, and up to 1.0% in hair products. Furthermore, zinc pyrithione is also allowed in a concentration up to 0.1% in leave-on hair products. More recently, it has been approved for use in rinse-off anti-dandruff hair care products in concentration from 1.0% to 2.0%. As such, zinc pyrithione (or pyrithione zinc) is commonly employed in zinc bar soaps or shampoos for treating and preventing itching, flaking and scaling of the scalp caused by dandruff or seborrhea, and available without prescription. In such bar soaps and shampoos (ZNP Bar®, DermaZinc Soap®, Head and Shoulders®), it is used with warm water to be massaged as a lather into the hair and scalp, being typically used once per day, or at least two times per week as directed by the user's physician.

In accordance with the present invention, and through clinical type observations, it has been determined that a topical application including pyrithione zinc is effective in significantly reducing the redness, flushing and inflammation associated with the chronic, incurable adult acne-like skin condition of Rosacea—and with skin erythema, in general.

The composition of the invention may comprise all pharmaceutical forms for administration including solutions, gels, lotions and creams—as well as ointments, foams, emulsions, micro-emulsions, milks, serums, aerosols, sprays, dispersions, micro-capsules and micro-particles thereof. Acceptable carriers for the pyrithione zinc are those suitable for topical applications for the skin, will not cause any safety or toxicity concerns, and will be compatible with the pyrithione zinc. One such carrier employed successfully with the pyrithione zinc has been found to be cocoa butter, an edible vegetable fat extracted from the cocoa bean. A ratio of about 3 parts cocoa butter to 1 part pyrithione zinc provided almost optimum results in effectiveness to decrease the erythema resulting from Rosacea. Also utilizable as soaps and cleansing bars, a 1% concentration of pyrithione zinc ingredient has been approved by the FDA for cover-the-counter sales—and thus more readily obtainable, and at a lesser cost than other prescription medications used to treat these skin conditions. Effective in most instances of Rosacea, more resistant situations can be resolved by increasing the pyrithione zinc concentrations to 2%, 3% or 4% levels; obtainable, however, only by way of dermatological prescription. Whether supplemented with known bases such as excipients, binders, lubricants and disintegrants—or with selected oily materials or emulsifying agents—the treatment with this composition has been noted to be effective in a simple one step application process. In fact, analysis has indicated that the composition of the invention with its use of pyrithione zinc can provide similar benefits for treating other skin complaints, conditions and afflictions—as with antibacterial agents, anti-acne agents, antiparasitic agents, antifungal agents, anti-inflammatory agents, and others. As such, with the pyrithione zinc, such other discreet erythema as acne and sunburn can be effectively reduced as well.

In usage, the composition of the invention is to be administered topically by a vehicle or carrier medium compatible with application to the skin. A useful regimen would be to a) mix 1% pyrithione zinc with 3 ml of cocoa butter cream; b) apply it to the area of concern; and c) leave the blended mixture for 2-3 minutes at the area of application. The composition will be seen to be highly effective in reducing the flushness and redness caused by Rosacea. In a [referred embodiment of the invention, the pyrithione zinc is employed as the sole active agent in the composition.

While there have been described what are considered to be preferred embodiments of the present invention, it will be readily appreciated by those skilled in the art that modifications can be made without departing from the scope of the teachings herein. For at least such reason, therefore, resort should be had to the claims appended hereto for a true understanding of the scope of the invention.

1. A method of treating erythema resulting from Rosacea in a subject in a one step application comprising administering topically to the skin of said subject a composition consisting of about 20% to about 30% of pyrithione zinc, wherein said pyrithione zinc is effective to decrease the erythema, and wherein said about 20% to about 30% of pyrithione zinc in said composition serves as the sole active ingredient of the composition for treating the erythema resulting from Rosacea.

2. The method of claim 1 wherein a pharmaceutical form for administering said composition is selected from solutions, gels, lotions, creams, ointments, foams, emulsions, micro-emulsions, milks, serums, aerosols, sprays, dispersions, micro-capsules and micro-particles thereof.

3. (canceled)

4. (canceled)

5. (canceled)

6. (canceled)

7. The method of claim 2 wherein said pharmaceutical form for administering said composition in said one step application consists of about 20% to about 30% of pyrithione zinc of 1% to about 4% concentration.

8. (canceled)

9. The method of claim 7 wherein said pharmaceutical form for administering said composition in said one step application consists of about 20% to about 30% of pyrithione zinc of 1% to about 4% concentration in a cocoa butter emulsion carrier.

10-13. (canceled)

14. A method of treating erythema resulting from Rosacea in a subject in a one step application comprising administering topically to the skin of said subject a pharmaceutical form of composition consisting of about 20% to about 30% of pyrithione zinc, wherein said 20% to about 30% of pyrithione zinc is effective to decrease the erythema, wherein said 20% to about 30% of pyrithione zinc serves as the sole active ingredient of the composition for treating the erythema resulting from Rosacea, and wherein said pharmaceutical form of composition once administered is left for 2-3 minutes at said skin area of administration.

15. The method of claim 14 wherein the pharmaceutical form for administering said composition is selected from solutions, gels, lotions, creams, ointments, foams, emulsions, micro-emulsions, milks, serums, aerosols, sprays, dispersions, micro-capsules and micro-particles thereof.

16. The method of claim 15 wherein said pharmaceutical form for administering said composition in said one step application consists of about 20% to about 30% of pyrithione zinc of 1% to about 4% concentration.

17. (canceled)

18. The method of claim 14 wherein said pharmaceutical form for administering said composition in said one step application consists of about 20% to about 30% of pyrithione zinc of 1% to about 4% concentration in a cocoa butter emulsion carrier.
